(19) United States
(12) Patent Application Publication
(10) Pub. No.: US 2025/0049981 A1
(43) Pub. Date: Feb. 13, 2025

(54) Wound Dressings and Methods of Making the Same

(71) Applicant: Solventum Intellectual Properties Company, Maplewood, MN (US)

(72) Inventors: Bryan A. Baker, Minneapolis, MN (US); Randilynn B. Christensen, Pine Springs, MN (US); Jason W. Bjork, Cottage Grove, MN (US); Saghi Saghazadeh, Apple Valley, MN (US); Kathleen L. Derrick, San Antonio, TX (US); Amy K. McNulty, Stillwater, MN (US); Kristine M. Kieswetter, San Antonio, TX (US)

(21) Appl. No.: 18/719,637

(22) PCT Filed: Dec. 15, 2022
(86) PCT No.: PCT/IB2022/062330

§ 371 Date: Jun. 13, 2024

Related U.S. Application Data
(60) Provisional application No. 63/428,472, filed on Nov. 29, 2022.
(60) Provisional application No. 63/265,597, filed on Dec. 17, 2021.

(51) Int. Cl. A61L 15/32; A61L 15/18; A61L 15/22; A61L 15/42

(52) U.S. Cl. CPC A61L 15/325; A61L 15/18; A61L 15/225; A61L 15/425

(57) ABSTRACT

An article and a method of making the article. The article can include a conformable matrix comprising a mixture of collagen, bioactive glass, and an additive selected from cellulose and/or alginate; wherein the article is a conformable wound dressing. Methods of making articles and methods of using articles to treat wounds are described.

### BACKGROUND

Collagen dressings are used as wound care products. These products are primarily derived from bovine collagen sources, particularly bovine skin, and processed via acid or enzymatic extraction methods into purified and largely type I collagen material. Collagen supports new tissue generation in a wound in part by attracting cells to the wound site, promoting cell adhesion, cell growth, and the production of new proteins. There is a need to provide better collagen dressings in forms useful for treating wounds.

### SUMMARY

A wound dressing, a method of making the wound dressing, and a method of treating a wound with the wound dressing are described. The wound dressing advantageously releases calcium ions that enhances healing.

In a first aspect, a wound dressing that is conformable is provided. The wound dressing comprises a conformable matrix that comprises a mixture comprising i) collagen, ii) bioactive glass, and ii) an additive comprising cellulose and/or alginate.

In a second aspect, a method of treating a wound is provided. The method comprising contacting an exposed surface of a wound with the wound dressing described in the first aspect.

In a third aspect, a method of making a wound dressing that contains a conformable matrix is provided. The method comprises forming a mixture comprising i) an acid solution, ii) collagen, iii) bioactive glass, and an additive comprising cellulose and/or alginate. The method further includes placing the mixture in a mold to form a cast mixture and then dehydrating the cast mixture to form the conformable matrix.

### DETAILED DESCRIPTION

Before any embodiments of the present disclosure are explained in detail, it is understood that the disclosure is not limited in its application to the details of use, construction, and the arrangement of components set forth in the following description. The present disclosure is capable of other embodiments and of being practiced or of being carried out in various ways that will become apparent to a person of ordinary skill in the art upon reading the present disclosure. Also, it is understood that the phraseology and terminology used herein is for the purpose of description and should not be regarded as limiting. The use of “including,” “comprising,” “containing”, or “having” and variations thereof herein is meant to encompass the items listed thereafter and equivalents thereof as well as additional items. It is understood that other embodiments may be utilized, and structural or logical changes may be made without departing from the scope of the present disclosure.

As used herein, the term “and/or” such as in the phrase “A and/or B” means A alone, B alone, or both A and B together.

A wound dressing that is conformable is provided. The term “conformable” means that the wound dressing is flexible and can be shaped to contours of a human or animal wound, enabling interaction (such as contact) of the wound dressing with a non-uniform surface found in or on different kinds of wounds.

The wound dressing comprises a conformable matrix that is a mixture that includes i) collagen, ii) bioactive glass, and ii) an additive comprising cellulose and/or alginate.

Any suitable sources of collagen can be used. It is typically Type I collagen and can be obtained from human, bovine, porcine, or other animal sources. Collagen can also be obtained from recombinant sources. Collagen can be obtained commercially as aqueous solutions, and the concentrations of these solutions may vary. Alternatively, collagen can be provided in lyophilized form and stored at very low temperatures. In some embodiments, collagen can be dissolved in an acid such as acetic acid. In some embodiments, the amount of collagen is at least 1 mg/mL and typically no greater than 120 mg/mL.

The collagen is often present in the conformable matrix in an amount ranging from 5 to 95 weight percent based on the total weight of the solid content of the conformable matrix. The amount can be at least 5, at least 10, at least 15, at least 20, at least 30, at least 40, at least 50, at least 55, at least 60, or at least 65 weight percent and up to 95, up to 90, up to 80, up to 70, up to 60, up to 50, up to 40, up to 30, up to 20, or up to 10 weight percent. In some embodiments, the amount of the collagen in the solid content of the conformable matrix is in a range of 40 to 95 weight percent, 50 to 95 weight percent, or 65 to 90 weight percent. In other embodiments, the amount of the collagen in the solid content of the conformable matrix is in a range of 30 to 50 weight percent, 20 to 30 weight percent, 10 to 20 weight percent, or 5 to 10 weight percent.

Any suitable sources of bioactive glass can be used in the preparation of the conformable matrix of the wound dressing. The bioactive glass may be melt-derived or sol-gel derived. A bioactive glass material suitable for the wound dressing and methods typically contains various inorganic oxides and/or inorganic fluorides such as those containing, for example, silicon, sodium, calcium, phosphorous, boron, potassium, and magnesium. The following composition, having a weight percent of each element in oxide or fluoride form in the range indicated, will provide bioactive glass compositions that may be used to form a bioactive glass.

### Example Bioactive Glass Composition

| Oxide | Percent weight range |
|---|---|
| SiO₂ | 0-86 |
| CaO | 4-35 |
| Na₂O | 0-35 |
| P₂O₅ | 2-15 |
| CaF₂ | 0-25 |
| B₂O3 | 0-75 |
| K₂O | 0-8 |
| MgO | 0-5 |

Divalent cations or ions that may optionally be present in any of the bioactive glasses include one or more of ferrous or ferric iron, alumina, cobalt, copper, magnesium, zinc, strontium, potassium, silver, barium, and titanium.

An exemplary bioactive glass is 45S5, which includes 46.1 mol % SiO₂, 26.9 mol % CaO, 24.4 mol % Na₂O, and 2.5 mol % P₂O₅. An exemplary borate bioactive glass is 45S5B1, in which the SiO₂ of 45S5 bioactive glass is replaced by B₂O₃. Other exemplary bioactive glasses include 58S, which includes 60 mol % SiO₂, 36 mol % CaO and 4 mol % P₂O₅, and S70C30, which includes 70 mol % SiO₂ and 30 mol % CaO. Other exemplary bioactive glasses include PSr40 which is 50% P₂O₅, 40% SrO, 10% Na₂O Mol %. Many such bioactive glasses can be obtained from Ceradyne (Saint Paul, MN, USA).

The bioactive glass that is included in the wound dressing can provide wound healing properties. For example, the bioactive glass can release soluble ions (such as Ca⁺², Na⁺, K⁺, and/or Mg⁺²) to a wound. Soluble ions can stimulate healing of a wound. Bioactive glass can also stimulate wound healing by modifying (increase or decrease) the pH of a wound environment. Modification of pH can in part reduce undesirable bacterial growth in the wound environment.

The bioactive glass included in conformable matrix is preferably not in the form of electrospun fibers or nanofibers. Rather, the bioactive glass that is used to prepare the conformable matrix is in the form of particles (e.g., a powder). Bioactive glass can be distributed throughout the conformable matrix of a wound dressings as discrete particles.

The bioactive glass in the solid content of the conformable matrix is usually in a range of 3 to 95 weight percent. The amount can be at least 3, at least 5, at least 10, at least 15, at least 20, at least 25, at least 30, at least 40, at least 50, at least 60, at least 70, at least 80, or at least 90 weight percent and up to 95, up to 90, up to 80, up to 70, up to 60, up to 50, up to 45, up to 40, up to 35, up to 30, up to 25, up to 20, up to 15, or up to 10 weight percent. The amount can be, for example, in a range of 3 to 10, 3 to 50, 5 to 35, 5 to 50, or 50 to 95 weight percent.

The weight ratio of collagen to bioactive glass (collagen weight: bioactive glass weight) in the conformable matrix is in a range of 1:12 to about 40:1. The ration can be at least 1:12, at least 1:2, at least 1:1, at least 1.5:1, at least 2:1, at least 3:1, at least 4:1, or at least 5:1 and up to 40:1, up to 30:1, up to 25:1, up to 20:1, up to 10:1, up to 9.5:1, up to 9:1, up to 5:1, up to 4:1, up to 3:1, or up to 2:1.

The mixture used to form the conformable matrix further includes an additive comprising cellulose and/or alginate. That is, the additive can include cellulose, alginate, or both cellulose and alginate.

The cellulose additive can be from any suitable source of cellulose and oxidized cellulose including oxidized regenerated cellulose (ORC). Oxidized cellulose can be prepared by oxidizing the hydroxyl groups of cellulose with an oxidizing agent such as dinitrogen tetroxide, hypochlorite, hydrogen peroxide, peracetic acid, or permanganate. In the oxidation process, at least a portion of the hydroxyl groups are oxidized to aldehyde, ketone, and/or carboxylic acid groups. The cellulose to be oxidized can be in either the regenerated form (e.g., rayon) or non-regenerated form. Oxidized cellulose can be prepared, for example, by a process described in U.S. Pat. No. 5,780,618 (Banker). Oxidized regenerated cellulose (ORC) can be prepared, for example, by a process described in U.S. Pat. No. 3,122,479 (Smith). ORC powder is commercially available, for example, as SURGICEL absorbable hemostat powder from Johnson & Johnson Company (New Brunswick, NJ, USA).

Cellulose, oxidized cellulose, and oxidized regenerated cellulose (ORC) can provide wound healing properties to the wound dressing. This healing can occur in part by modifying the pH of the wound environment (thereby reducing bacterial growth), increasing moisture content of the wound environment, reducing elastase activity, and/or improving the hemostatic properties of the wound.

The cellulose in the solid content of the conformable matrix is usually in a range of 5 to 60 weight percent. The amount can be at least 5, at 10, at least 15, at least 20, at least 25, at least 30, at least 35, or at least 40 weight percent and up to 60, up to 55, up to 50, up to 45, up to 40, up to 35, or up to 30 weight percent. The amount can be, for example, in a range of 15 to 50, 20 to 50, 30 to 50, or 40 to 50 weight percent.

The additive can include alginate. Any suitable source of alginate can be used. Alginates are anionic, polysaccharide biopolymers constructed from alpha-L-glucuronic acid and beta-D-mannuronic acid monomers. In the biopolymers, the monomers are covalently 1,4-linked in monomer blocks. Alginates can be extracted from brown seaweeds or produced from some Pseudomonas and Azotobacter strains. The alginate is often used in the form of a salt such as sodium alginate or calcium alginate.

Like cellulose, alginate can provide wound healing properties to the wound dressing. This healing can occur in part by increasing the moisture content of the wound environment, absorbing wound exudate, improving the hemostatic properties of the wound, and/or reducing bacterial growth.

The alginate in the solid content of the conformable matrix usually in a range of 3 to 20 weight percent. The amount can be at least 3, at least 5, at least 10, or at least 15 weight percent and up to 20, up to 15, or up to 10 weight percent. The amount, for example, can be in a range of 3 to 15, 5 to 20, or 5 to 15 weight percent.

When the conformable matrix of the wound dressing contains oxidized cellulose or ORC as the additive, the wound dressing can have a pH value in a range of 5 to 9.9. The pH can be at least 5, at least 6, at least 7, or at least 8 and up to 9.5, up to 9, up to 8.7, up to 8.5, up to 8, up to 7.5, or up to 7, of greater than 5, greater than 6, greater than 7, or greater than 8. For example, the pH range can be from 6 to 9.5, 7 to 9.5, 7.5 to 9.5, 7 to 9, 7 to 8.5, 6 to 8.5, or 5 to 8.5.

When the conformable matrix contains alginate as the additive, the wound dressing can have a pH value in a range of 7.3 to 11.5. The pH can be at least 7.3, at least 7.5, at least 8, at least 8.3, or at least 8.5 and up to 11.5, up to 11, up to 10.5, up to 10, up to 9.5, or up to 9. For example, the pH range can be from 7.3 to 11.5, 7.3 to 11, 7.5 to 11, 8 to 11, 8 to 10.5, or 8 to 9.5.

Not to be bound by theory, altering pH may help facilitate the healing of wound tissue by reducing enzymatic activity and/or inhibiting bacterial growth.

The thickness of a conformable matrix is typically about 0.8 millimeters (mm) to about 10 mm. For example, the thickness can be at least 1, at least 2, at least 3, or at least 5 millimeters and up to 10, up to 8, up to 6, up to 5, or up to 4 mm.

In some embodiments, the conformable matrix is porous.

The wound dressing can further include an optional substrate. The substrate can be selected from foam, mesh, netting, woven, nonwoven, cotton, cellulose fabrics, perforated film, hydrocolloid, hydrogel, polymers with inherent porosity, pressure sensitive adhesive, and combination of thereof.

If the wound dressing includes a substrate, the substrate can be or include an absorbent material. Exemplary absorbent material can include a film, fabric, or porous materials made from viscose, rayon, alginate, gauze, biopolymers, polyurethane, biodegradable polymers, or hydrophilic polymers. These substrates can further include a metal compound (e.g., a metal compound containing silver, copper, zinc, can combinations thereof) such as those described, for example, in U.S. Pat. No. 7,745,509 (Burton et al.). The absorbent materials can be manufactured of any suitable materials including, but not limited to, woven or nonwoven cotton or rayon or netting and perforated film made from nylon, polyester or polyolefins. Absorbent pads can be used as the absorbent material and can be useful for containing optional substances such as drugs for transdermal drug delivery, chemical indicators to monitor hormones or other substances in a patient, and the like.

The absorbent material may include a hydrocolloid composition, including the hydrocolloid compositions described, for example, in U.S. Pat. No. 5,622,711 (Chen) and 5.633.010 (Chen). The hydrocolloid absorbent can include, for example, a natural hydrocolloid, such as pectin, gelatin, or carboxymethylcellulose (CMC) (available from Aqualon Corp., Wilmington, DE, USA), a semi-synthetic hydrocolloid such as cross-linked carboxymethylcellulose commercially available from FMC Corp. (Philadelphia, PA, USA) under the trade designation Ac-Di-Sol, a synthetic hydrocolloid such as cross-linked polyacrylic acid (PAA) commercially available from B.F. Goodrich (Brecksville. OH, USA) under the trade designation CARBOPOL No. 974P, or a combination thereof. Absorbent materials can be manufactured of other synthetic and natural hydrophilic materials including polymer gels and foams. In some embodiments, the substrate is a hydrocolloid polymer.

The wound dressing as well as the conformable matrix of the wound dressing can be in any suitable physical form, such as a sheet (i.e. film), a gel, sponge, scaffold, or foam.

If desired, the wound dressing can be disposed on or within a carrier layer. In some embodiments, the carrier can be a carrier layer disposed on a major surface of the wound dressing. A carrier layer is typically disposed on the opposing major surface as the wound-facing surface.

In some embodiments, carrier layer is a release liner. The release liner carrier may be disposed on the opposing major surface of both major surfaces (not shown) such that the wound dressing (and the conformable matrix) is a sheet (i.e., film), a gel, sponge, scaffold, or foam positioned between the release liner layers.

Various release liners are known such as those made of paper (e.g., Kraft paper), polyolefin film (e.g. polyethylene, polypropylene, or copolymers thereof) or polyester film. The paper or polymeric film is often coated with a release agent such as a fluorochemical or silicone. For example, U.S. Pat. No. 4,472,480 (Olson) describes low surface energy perfluorochemical liners. Examples of commercially available silicone coated release papers include those commercially available under the trade designation POLYSLIK, silicone release papers available from Rexam Release (Bedford Park. IL, USA), and silicone release papers commercially available from Loparex (Willowbrook, IL, USA). Other non-limiting examples of such release liners commercially available include siliconized polyethylene terephthalate films commercially available from H. P. Smith Co. and fluoropolymer coated polyester films (release liners) commercially available from 3M Company (Saint Paul, MN, USA) under the trade designation SCOTCHPAC.

In other embodiments, the carrier layer may comprise a variety of other (e.g. flexible and/or conformable) carrier materials such as polymeric films and foams as well as various nonwoven and woven fibrous materials, such as gauze. In some embodiments, the carrier layer is absorbent, such as an absorbent foam. In other embodiments, the carrier layer is non-absorbent, such as a polymeric film.

The conformable matrix can optionally further include a plasticizing agent, for example, glycerol, diglycerol, triglycerol, xylitol, mannitol, or C3-C24 alkane diols (e.g., butane diol and propane diol) to improve the hydration of the wound dressing. Additionally, the plasticizing agent may improve the conformability and handleability of the wound dressing by reducing the brittleness and allowing deformation before cracking or tearing. The plasticizing agent is often glycerol.

In some embodiments, the amount of plasticizing agent in the conformable matrix is in a range of 0.01 to 1.5 weight percent based on the total weight of the solid content of the conformable matrix. The amount can be at least 0.01, at least 0.02, at least 0.03, at last 0.05, at least 0.1, at least 0.2, at least 0.3, at least 0.5, or at least 1 weight percent and up to 1.5, up to 1.3, up to 1.2, up to 1.1, up to 1.0, up to 0.5, or up to 0.1 weight percent.

The wound dressing of present application can increase expression of vascular endothelial growth factor (VEGF) and hepatocyte growth factor (HGF). These two growth factors can significantly contribute to wound healing. VEGF is known to increase the angiogenesis and HGF has been shown to improve epidermal keratinocytes migration resulting in enhanced reepithelialization. Angiogenesis plays a crucial role in wound healing by forming new blood vessels from preexisting vessels by invading the wound clot and organizing into a microvascular capillary network throughout the granulation tissue. Wound reepithelialization is also key in the goal of wound closure. The epithelialization process can be stalled by a number of factors, all of which must be resolved before wound healing can move forward. By promoting angiogenesis and reepithelialization, the wound dressing of present application can increase the success rate of a wound to heal.

In another aspect, a method of making a wound dressing that includes a conformable matrix is provided. The method includes forming a mixture comprising i) an acid solution, ii) collagen, iii) an additive comprising cellulose and/or alginate, and iv) bioactive glass. The method further includes placing the mixture in a mold to form a cast mixture and dehydrating the cast mixture to form the conformable matrix.

The acid solution that is used in the mixture is often either acidic acid or hydrochloric acid. The acid concentration can be from about 0.005 M to 1 M or about 0.01 M to 0.1 M. In some embodiments, a plasticizing agent such as glycerol is added to the acid solution.

The collogen is often added first to the acid solution. Then the additive comprising cellulose and/or alginate is added to the acid solution. The bioactive glass is then added to form the mixture. After mixing, the conformable matrix is formed by molding to form a cast mixture and then dehydrating the cast mixture. The dehydrating step may be conducted by any suitable means, such as, freeze-drying, air-drying, oven drying, critical point drying, or combination thereof.

In some embodiments of the method of making the wound dressing further includes disposing the wound dressing on or within a carrier.

In yet another aspect, a method of treating a wound with a wound dressing is provided. The wound dressing is the same as described above. The method of treating comprises covering at least a portion of the wound with the wound dressing. In some embodiments, the method of treating increases the pH of the wound environment. In some embodiments, the method of treating decreases the pH of the wound environment. In some embodiments, the method of treating increases the ionic content of the wound environment as increasing the amount of calcium ion. In some embodiments, the method of treating comprises releasing calcium ions from the wound dressing to the wound environment. The wound environment can be the wound bed. The wound environment can be an exposed wound surface.

The wound treated by this method can be an open wound of the skin that exposes underlying body tissue. Open wounds that can be treated by the method include acute wounds and chronic wounds. Open wounds that can be treated by the method include wounds to the skin from trauma (for example avulsions, incisions, and lacerations); wounds to the skin from pressure (for example pressure ulcers); and wounds to the skin from disease (for example venous ulcers, diabetic foot ulcers, and diabetic leg ulcers).

The following working examples are intended to be illustrative of the present disclosure and not limiting.

### EXAMPLES

Objects and advantages of this disclosure are further illustrated by the following examples, but the particular materials and amounts thereof recited in these examples, as well as other conditions and details, should not be construed to unduly limit this disclosure.

### Materials

Collagen type I from bovine calf skin (product #C857) was obtained from Elastin Products Company, Owensville, MO.

Glycerol was obtained from the Sigma-Aldrich Corporation, St. Louis, Mo.

Phosphate buffered saline (PBS, 1×) was obtained from Thermo Fisher Scientific, Waltham, MA.

Bioactive Glass A (BG-A) was obtained from the 3M Corporation (Maplewood, MN) as a powder with the following composition: Na₂O (9.3 weight %). CaO (19.7 weight %). P₂O₅ (71.0 weight %). The reported glass transition temperature (Tg) was 418.5° C.

Bioactive Glass A (BG-A) was obtained from the 3M Corporation as a powder with the following composition: SiO₂ (45 weight %). Na₂O (24.5 weight %). CaO (24.5 weight %). P₂O₅ (6 weight %). The reported glass transition temperature (Tg) was 528.6° C.

Bioactive Glass B (BG-B) was obtained from the 3M Corporation as a powder with the following composition: Na₂O (5.5 weight %). CaO (18.5 weight %). P₂O₅ (3.7 weight %). K₂O (11.1 weight %), MgO (4.6 weight %). B₂O₃ (56.6 weight %). The reported glass transition temperature (Tg) was 518.8° C.

PROMOGRAN Matrix Wound Dressing (55% collagen and 45% oxidized regenerated cellulose) was obtained from the 3M Company, St. Paul, MN. FIBRACOL Plus Collagen Wound Dressing with Alginate (90% collagen and 10% calcium alginate) was obtained from the 3M Company.

Human dermal fibroblast cells (HDFa) cells (product no. PCS-201-012, obtained from ATCC, Manassas, MA) were maintained in culture using Dulbecco's Modified Eagle Medium (DMEM, product no. 10567014, Thermo Fisher Scientific) supplemented with 10% by volume fetal bovine serum (FBS, product no. 26140079, Thermo Fisher Scientific) at 37° C. in a 5% CO₂ humidified incubator. The cells were passaged according to the manufacturer's protocol, cultured in 12 well plates and allowed to reach confluence.

Human umbilical vein endothelial cells (HUVECs, product no. C0035C. obtained from Thermo Fisher Scientific) were maintained in culture using human large vessel endothelial cell basal medium (product no. M200500, obtained from Thermo Fisher Scientific) supplemented with 10% by volume low serum growth supplement (LSGS, product no. S00310, obtained from Thermo Fisher Scientific) at 37° C. in a 5% CO₂ humidified incubator. Cells were passaged according to the manufacturer's protocol.

### Examples 1-6. Preparation of Collagen-Bioactive Glass-ORC Conformable Matrix

A sample of PROMOGRAN Matrix Wound Dressing was cut into rectangular sections (approximately 1.3 cm by 6.5 cm) and 2.0 g of the cut sections were suspended in 200 mL of 0.05 M acetic acid. The suspension was maintained at room temperature for 5 minutes and then blended with an electric hand mixer for about 10-30 seconds. Next, either 200 mg, 400 mg, or 1 g of either Bioactive Glass A or Bioactive Glass B was mixed into the suspension. Mixing was done using the electric hand mixer for about 10-30 seconds. The resulting suspension was cast into a 6.3 cm inner diameter plastic tray (pre-treated with Rocket Release #E302 food grade release agent, obtained from Stoner Molding Solutions. Quarryville, PA) and then frozen at 0° C. until solid. The frozen suspension was lyophilized to provide the collagen-bioactive glass-ORC product as a conformable, porous matrix. Lyophilizations were conducted using a VirTis Advantage Plus EL-85 Freeze Dryer (SP Scientific, Warminster, PA). Summary descriptions of the products are provided in Table 1.

### Comparative Example A. Collagen-ORC Matrix without Bioactive Glass

A collagen-ORC product without bioactive glass was prepared by not including the step of adding bioactive glass in the procedure described for Examples 1-6. A summary description of the product is provided in Table 1.

TABLE 1

| | | Amount of | | Calculated weight % |
|---|---|---|---|---|
| | | Bioactive Glass | | of Bioactive Glass in |
| | Bioactive | added to Acetic | | Porous Matrix after |
| Product | Glass | Acid | | Lyophilization |
| Example 1 | BG-A | 200 | mg | 9.1% |
| Example 2 | BG-A | 400 | mg | 16.7% |
| Example 3 | BG-A | 1 | g | 33.3% |
| Example 4 | BG-B | 200 | mg | 9.1% |
| Example 5 | BG-B | 400 | mg | 16.7% |
| Example 6 | BG-B | 1 | g | 33.3% |

| | | | |
|---|---|---|---|
| Comparative | None | None | 0% |
| Example A | | | |

### Examples 7-12. Preparation of Collagen-Bioactive Glass-Alginate Conformable Matrix

A sample of FIBRACOL Plus Collagen Wound Dressing with Alginate was cut into rectangular sections (approximately 1.3 cm by 6.5 cm) and 2.0 g of the cut sections were suspended in 200 mL of 0.05 M acetic acid. The suspension was maintained at room temperature for 5 minutes and then blended with an electric hand mixer for about 10-30 seconds. Next, either 200 mg, 400 mg, or 1 g of either Bioactive Glass A or Bioactive Glass B was mixed into the suspension. Mixing was done using the electric hand mixer for about 10-30 seconds. The resulting suspension was cast into a 6.3 cm inner diameter plastic tray (pre-treated with Rocket Release #E302 food grade release agent, obtained from Stoner Molding Solutions) and then frozen at 0° C. until solid. The frozen suspension was lyophilized to provide the collagen-alginate-bioactive glass product as a conformable, porous matrix. Lyophilizations were conducted using a VirTis Advantage Plus EL-85 Freeze Dryer (SP Scientific). Summary descriptions of the products are provided in Table 2.

### Comparative Example B. Collagen-Alginate Matrix without Bioactive Glass

A collagen-alginate product without bioactive glass was prepared by not including the step of adding bioactive glass in the procedure described for Examples 7-12. A summary description of the product is provided in Table 2.

TABLE 2

| | | Amount of | | Calculated weight % |
|---|---|---|---|---|
| | | Bioactive Glass | | of Bioactive Glass in |
| | Bioactive | added to Acetic | | Porous Matrix after |
| Product | Glass | Acid | | Lyophilization |
| Example 7 | BG-A | 200 | mg | 9.1% |
| Example 8 | BG-A | 400 | mg | 16.7% |
| Example 9 | BG-A | 1 | g | 33.3% |
| Example 10 | BG-B | 200 | mg | 9.1% |
| Example 11 | BG-B | 400 | mg | 16.7% |
| Example 12 | BG-B | 1 | g | 33.3% |

| | | | |
|---|---|---|---|
| Comparative | None | None | 0% |
| Example B | | | |

### Example 13. Measurement of Ca²⁺ Ion Release for Collagen-Bioactive Glass-ORC Matrix Samples and Collagen-Bioactive Glass-Alginate Matrix Samples

Sample discs (50 mm diameter) for testing were punched from the articles of Examples 1-6, 7-8, 10-11 and Comparative Examples A-B. Each sample disc was individually placed in a plastic vial (i.e., one disc per vial) containing 5 mL of EPILIFE medium (Thermo Fisher Scientific). Each vial was capped, and then gently agitated (using a laboratory rocker table) at 4° C. for 24 hours. Control Samples A-G were prepared by mixing a sample of either BG-A or BG-B powder (in concentrations of either 0, 1, 10, or 100 mg/mL) with 5 mL of EPILIFE medium in a plastic vial (i.e., one powder sample per vial) and maintaining the mixture at 4° C. for 24 hours. Next, each vial was warmed to room temperature and the Ca²⁺ concentration (micromolar) of the liquid medium in the vial was measured using a calcium ion probe (Mettler Toledo Ion Selective Electrode #51344703, Mettler Toledo Company, Columbus, OH) with an ionic strength adjuster (ISA) solution provided by the manufacturer according to the manufacturer's instructions. For each example and comparative example article, three separate discs were prepared and tested with the measured Ca²⁺ concentrations reported as the average value (n=3). For the Control Samples A-G, the Ca²⁺ concentrations were reported based on a single measurement (n=1). The pH of the liquid medium was measured using a calibrated ACCUMET AE150 PH meter (Thermo Fisher Scientific). A single pH measurement was conducted for each article and control (n=1). The results are provided in Tables 3-5.

For Tables 3-5, the bioactive glass concentration in each test vial was determined according to the following equation:

$$Bioactive Glass ( \frac{mg}{mL} ) = \frac{[ ( weight of disc ) \times ( wt . \% of bioactive glass in sample ) ]}{volume of EPILIFE medium ( mL )}$$

The data in Tables 3-5 shows that significantly more Ca²⁺ ions were released from the conformable, porous matrix articles of the examples than from the Control Samples based on the concentration (mg/mL) of bioactive glass in the test sample.

TABLE 3
pH and Ca²⁺ Concentration Measurements using Control Samples

| | | Bioactive Glass | | Ca²⁺ Conc. |
|---|---|---|---|---|
| | Bioactive | Concentration | pH of Liquid | in Liquid Medium |
| | Glass | (mg/mL) | Medium | (micromolar) (n = 1) |
| Control Sample A | None | 0 | 7.7 | 48 |
| Control Sample B | BG-A | 1 | 8.3 | 542 |
| Control Sample C | BG-A | 10 | 8.9 | 841 |
| Control Sample D | BG-A | 100 | 9.7 | 226 |
| Control Sample E | BG-B | 1 | 8.4 | 1246 |
| Control Sample F | BG-B | 10 | 8.8 | 2693 |
| Control Sample G | BG-B | 100 | 9.1 | 2858 |

TABLE 4
pH and Ca²⁺ Concentration Measurements
using Collagen-Bioactive Glass-ORC Matrix Samples

| | | Bioactive Glass | | Average Ca²⁺ Conc. |
|---|---|---|---|---|
| | Bioactive | Concentration | pH of Liquid | in Liquid Medium |
| Disc from | Glass | (mg/mL) | Medium | (micromolar) (n = 3) |
| Comparative | None | 0 | 4.1 | 86 |
| Example A | | | | |
| Example 1 | BG-A | 4.7 | 6.9 | 6778 |
| Example 2 | BG-A | 8.4 | 7.5 | 6778 |
| Example 3 | BG-A | 14.6 | 7.8 | 15694 |
| Example 4 | BG-B | 4.2 | 6.6 | 5423 |
| Example 5 | BG-B | 9.3 | 7.6 | 8334 |
| Example 6 | BG-B | 16.7 | 8.3 | 9590 |

TABLE 5
pH and Ca²⁺ Concentration Measurements using
Collagen-Bioactive Glass-Alginate Matrix Samples

| | | Bioactive Glass | | Average Ca²⁺ Conc. |
|---|---|---|---|---|
| | Bioactive | Concentration | pH of Liquid | in Liquid Medium |
| Disc from | Glass | (mg/mL) | Medium | (micromolar) (n = 3) |
| Comparative | None | 0 | 7.9 | 4925 |
| Example B | | | | |
| Example 7 | BG-A | 4.7 | 8.0 | 9617 |
| Example 8 | BG-A | 9.8 | 8.0 | 18659 |
| Example 10 | BG-B | 4.7 | 8.4 | 8973 |
| Example 11 | BG-B | 9.8 | 8.5 | 14057 |

### Example 14

Sample discs (50 mm diameter) for testing were punched from the articles of Examples 1-6 and Comparative Example A and weighed. Each sample disc was individually placed in a plastic vial (i.e., one disc per vial) and Dulbecco's Modified Eagle Medium (DMEM) supplemented with 10% by volume fetal bovine serum (FBS) was added to a concentration of 5 mg/mL (mg of disc to mL of supplemented DMEM). Each vial was capped, and then gently agitated (using a laboratory rocker table) at 4° C. for 48 hours. Next, each vial was warmed to room temperature and the supernatant media was filtered using a Millex-GP 0.2 micron filter (product no. SLMPL25SS, MilliporeSigma, Burlington, MA). Each filtered sample of supernatant media was diluted 10-fold with a solution of DMEM that contained 10% by volume fetal bovine serum (FBS).

The cell culture medium (DMEM with 10% FBS) was aspirated from the wells of freshly prepared HDFa culture plates (culture plates prepared as described above). The wells were washed 3 times with fresh 1× PBS. Following the final wash step, aliquots (1 mL) of the diluted supernatant media samples were added to the wells of the plate with a single supernatant media sample added to an individual well (i.e., there was one supernatant media sample added per well). Negative Control wells were also prepared by adding a 1 mL aliquot of fresh DMEM that did not contain FBS and that had not been exposed to a sample disc. A total of 4 wells were prepared for each test, comparative, and control sample (4 replicates). The plates were incubated at 37° C. in a 5% CO₂ humidified incubator for 5 days.

Supernatant media (1 mL) was then extracted from each well and analyzed for hepatocyte growth factor (HGF) and vascular endothelial growth factor (VEGF) content using an Ella Automated Immunoassay System (Bio-techne, Minneapolis, MN) with VEGF and HGF Simple Plex cartridges (Bio-techne) according to the manufacturer's instructions. The mean concentrations (in picograms/mL (pg/mL) with calculated standard deviation) of HGF and VEGF secreted from HDFa cells in a well are reported in Table 6.

TABLE 6

| | Mean HGF Concentration | | Mean VEGF Concentration | |
|---|---|---|---|---|
| | Per Well | | Per Well | |
| | | Standard | | Standard |
| Disc from | pg/mL | Deviation | pg/mL | Deviation |
| Example 1 | 430.3 | 25.3 | 1105.7 | 41.1 |
| Example 2 | 1472.3 | 79.9 | 1430.3 | 84.7 |
| Example 3 | 580.7 | 36.6 | 1073.3 | 69.0 |
| Example 4 | 279.7 | 13.8 | 1570.3 | 127.4 |
| Example 5 | 341.0 | 29.6 | 2913.7 | 623.5 |
| Example 6 | 62.4 | 9.2 | 1411.7 | 22.7 |
| Comparative | 166.0 | 8.7 | 1059.3 | 30.6 |
| Example A | | | | |
| Negative | 132.3 | 8.1 | 259.0 | 9.6 |
| Control | | | | |

### Example 15

The same procedure as described in Example 14 was followed with the exception that sample discs (50 mm diameter) for testing were punched from the articles of Examples 7, 8 and Comparative Example B with each sample disc individually placed in a plastic vial (i.e., one disc per vial) and diluted to a concentration of 10 mg/mL with DMEM. The supernatant media extracted from each well was analyzed for VEGF. The mean concentration of VEGF secreted from HIDFa cells in a well are reported in Table 7.

TABLE 7

Mean VEGF Concentration
Per Well

| | | Standard |
|---|---|---|
| Disc from | pg/mL | Deviation |
| Example 7 | 9630.7 | 551.7 |
| Example 8 | 7771.0 | 376.5 |
| Comparative | 2558.7 | 469.0 |
| Example B | | |
| Negative | 411.2 | 32.3 |
| Control | | |

### Example 16

Sample discs (50 mm diameter) for testing were punched from the articles of Examples 2, 5 and Comparative Example A. For each sample disc, a single disc was added to a vial and diluted to a concentration of 10 mg/mL with human large vessel endothelial cell basal medium supplemented with 10% by volume low serum growth supplement (LSGS) and the immersed disc was incubated at 4° C. for 24 hours. Each vial was then warmed to room temperature and the supernatant media was filtered using a Millex-GP 0.2 micron filter. Each filtered sample of supernatant media obtained from a disc sample was diluted 10-fold with human large vessel endothelial cell basal medium supplemented with 10% by volume low serum growth supplement (LSGS). A Negative Control sample was also prepared by following the same procedure with the exceptions that human large vessel endothelial cell basal medium without LSGS supplement was used and a sample disc was not added to the vial.

Tissue culture plates for assays were prepared using 48 well culture plates that were coated by adding 200 microliters of GELTREX hESC-qualified, ready to use, reduced growth factor basement membrane matrix (product no. A1569601, obtained from Thermo Fisher scientific) to each well. Coated plates were maintained at room temperature for 1 hour and then the supernatant liquid was removed from each well by aspiration with a micropipette. Individual aliquots of HUVECs (each aliquot prepared as 30.000 cells in 200 microliters of a single diluted sample of supernatant media obtained from a disc or control sample) were immediately added to the coated wells of a plate. A total of 8 wells were prepared for each test, comparative, and control sample (8 replicates). The plates were incubated at 37° C. in a 5% CO₂ humidified incubator. After 6 hours, brightfield images of the wells were taken with a Lionheart-FX automated microscope (Agilent Technologies. Santa Clara. CA) at 4× magnification and analyzed using ImageJ software (National Institutes of Health, Bethesda. MD) with the “Angiogenesis Analyzer for ImageJ plug-in tool”. The cells in the images were analyzed for the number of Master Junctions. Master Segments. and Isolated Elements observed according to the procedures described in “Angiogenesis Analyzer for ImageJ—A Comparative Morphometric Analysis of “Endothelial Tube Formation Assay” and “Fibrin Bead Assay””. Carpentier, et. al., Scientific Reports, 10, 11568 (2020), doi.org/10.1038/s+1598-020-67289-B.

In the procedure, larger numbers of Master Segments and Master Junctions and lower numbers of Isolated Elements represent greater pro-angiogenic cell activity. “Junctions” indicate the multi-intersection junctions with three or more furcated branches in the angiogenic structures. If furcated branches of the junction are exclusively connected with other capillary structures and thereby without extremities, this junction is called a “Master Junction”. “Segments” represent portions of an angiogenic capillary with two ends connected to two junction points. If these two junctions are “Master Junctions”, this segment is called a “Master Segment”. Elements limited by two extremities were classified as “Isolated Elements”.

The results are reported in Table 8 as the mean value (with calculated standard deviation) from 8 replicates.

TABLE 8

| | Number of Master | | Number of Master | | Number of Isolated | |
|---|---|---|---|---|---|---|
| | Segments Per Image | | Junctions Per Image | | Elements Per Image | |
| | | Standard | | Standard | | Standard |
| Disc Used | Mean | Deviation | Mean | Deviation | Mean | Deviation |
| Example 2 | 60.1 | 1.8 | 45.3 | 0.9 | 3.8 | 0.9 |
| Example 5 | 106.1 | 5.1 | 50.6 | 3.1 | 3.7 | 1.0 |
| Comparative | 54.1 | 5.7 | 39.4 | 2.8 | 2.0 | 0.9 |
| Example A | | | | | | |
| Negative Control | 32.9 | 1.8 | 9.8 | 2.0 | 14.5 | 1.9 |

All references and publications cited herein are expressly incorporated herein by reference in their entirety into this disclosure. Illustrative embodiments of this disclosure are discussed and reference has been made to possible variations within the scope of this disclosure. For example, features depicted in connection with one illustrative embodiment may be used in connection with other embodiments of the disclosure. These and other variations and modifications in the disclosure will be apparent to those skilled in the art without departing from the scope of the disclosure, and it should be understood that this disclosure is not limited to the illustrative embodiments set forth herein. Accordingly, the disclosure is to be limited only by the claims provided below and equivalents thereof.

11. A wound dressing, comprising:
a conformable matrix, comprising:
collagen;
bioactive glass; and
an additive comprising at least one of cellulose or alginate.

2. The wound dressing of claim 1, wherein the cellulose is oxidized cellulose.

3. The wound dressing of claim 2, wherein the oxidized cellulose is oxidized regenerated cellulose.

4. The wound dressing of claim 1, wherein the alginate is selected from the group consisting of sodium alginate and calcium alginate.

5. The wound dressing of claim 1, wherein the conformable matrix further comprises glycerol.

6. The wound dressing of claim 1, further comprising a carrier, wherein the conformable matrix is disposed on or within the carrier.

7. The wound dressing of claim 1, wherein the conformable matrix has a pH value more than 7.3.

8. The wound dressing of claim 1, wherein the conformable matrix is in the form of a material selected from the group consisting of a gel, a sponge, a scaffold, a foam, a sheet, and a film.

9. A method of treating a wound on a patient, the method comprising:
contacting a wound dressing with an exposed surface of the wound, wherein the wound dressing comprises a conformable matrix, comprising:
collagen;
bioactive glass; and
an additive comprising at least one of cellulose or alginate; and
leaving the wound dressing on the wound for a desired amount of time to treat the wound.

10. The method of claim 9, wherein the method comprises releasing Ca²⁺ ions from the wound dressing to the wound.

11. A method of making a wound dressing comprising a conformable matrix, the method comprising:
forming a mixture, comprising:
an acid solution;
collagen;
an additive comprising at least one of cellulose or alginate; and
bioactive glass;
placing the mixture in a mold to form a cast mixture; and
dehydrating the cast mixture to form the conformable matrix.

12. The method of claim 11, wherein the collagen is mixed with the acid solution prior to the addition to the mixture of the additive.

13. The method of claim 11, wherein dehydrating the cast mixture comprises performing a dehydration process selected from the group consisting of freeze-drying, air-drying, oven drying, critical point drying, and combinations thereof.

14. The method of claim 11, wherein the acid solution comprises an acid selected from the group consisting of acetic acid and hydrochloric acid.

15. The method of claim 14, wherein the concentration of the acid in the acid solution is 0.005 M to 1 M.
